(19) United States
(12) Patent Application Publication
(10) Pub. No.: US 2026/0327721 A1
(43) Pub. Date: Oct. 22, 2026

(54) EVALUATION METHOD FOR EFFECTS OF INSECT-RESISTANT TRANSGENIC CROPS ON DIVERSITY OF SYMBIOTIC BACTERIA IN TARGET AND NON-TARGET PESTS

(71) Applicant: Cotton Research Institute of Chinese Academy of Agricultural Sciences, Anyang (CN)

(72) Inventors: Xiangliang REN, Anyang (CN); Yan MA, Anyang (CN); Peng WANG, Anyang (CN); Yongpan SHAN, Anyang (CN); Hongyan HU, Anyang (CN); Junyu LUO, Anyang (CN); Yajie MA, Anyang (CN); Dan WANG, Anyang (CN); Changcai WU, Anyang (CN); Xianpeng SONG, Anyang (CN)

(21) Appl. No.: 19/318,758

(22) Filed: Sep. 4, 2025

(30) Foreign Application Priority Data
Apr. 21, 2025 (CN) 202510500274.5

(51) Int. Cl. C12Q 1/689; C12Q 1/6851; C12Q 1/686; C12Q 1/6869

(52) U.S. Cl. CPC C12Q 1/689; C12Q 1/6851; C12Q 1/686; C12Q 1/6869

(57) ABSTRACT

An evaluation method for effects of insect-resistant transgenic crops on diversity of symbiotic bacteria in target and non-target pests is suitable for insect-resistant transgenic crops with various construction methods. With the help of high-throughput sequencing technology, it can overcome the shortcomings of traditional cultivation technology, reduce workload, and shorten evaluation cycle. The specific steps include planting of transgenic crops, selection of experimental pests, treatment and collection of pest samples, extraction and sequencing of sample genomic deoxyribonucleic acid (DNA), diversity analysis, and quantitative verification of key bacterial groups. This method can comprehensively detect the diversity of symbiotic bacteria in pests, provide scientific basis for safety evaluation of insect-resistant transgenic crops, and provide reference for ecological environment effect evaluation of other transgenic crops.

### CROSS-REFERENCE TO RELATED APPLICATION

This application claims priority of Chinese Patent Application No. 202510500274.5, filed on Apr. 21, 2025, the entire contents of which are incorporated herein by reference.

### TECHNICAL FIELD

The present disclosure relates to the technical field of environmental safety evaluation of biotechnology products, and in particular to an evaluation method for the effects of insect-resistant transgenic crops on the diversity of symbiotic bacteria in target and non-target pests.

### BACKGROUND

Insect-resistant transgenic crops introduce foreign insect-resistant genes (such as Bacillus thuringiensis (Bt) gene) into crop genomes through genetic engineering technology, the insect-resistant transgenic crops can express proteins with insecticidal activity, thus effectively resisting pests. Since the first commercial planting of insect-resistant transgenic crops in 1996, the planting area has expanded year by year, and now the insect-resistant transgenic crops have been widely used in many countries around the world. Typical insect-resistant transgenic crops include insect-resistant cotton, insect-resistant corn and insect-resistant rice. These crops have played a significant role in reducing the use of chemical pesticides, reducing production costs, and improving crop yield and quality. However, with the expansion of cultivation scale, the potential impact on farmland ecosystems has also attracted considerable attention.

There are a large number of symbiotic bacteria in pests, and these symbiotic bacteria form a complex symbiotic relationship with pests. Symbiotic bacteria play a key role in many physiological processes including growth and development, reproduction, nutrient metabolism, and immune defense of pests. In addition, symbiotic bacteria of pests are closely related to the ecological adaptability of pests. Symbiotic bacteria can affect pests' ability to adapt to the environment and help pests survive and multiply in different ecological environments. Therefore, the diversity changes of symbiotic bacteria of pests have a profound impact on their biological characteristics and ecological behavior.

At present, there is a lack of systematic and comprehensive evaluation method for the effects of insect-resistant transgenic crops on the diversity of symbiotic bacteria in pests. Most of the existing evaluation methods use traditional culture methods to analyze the diversity of symbiotic bacteria. This method can only detect symbiotic bacteria that can grow on artificial medium, but cannot identify those that are difficult or unculturable, resulting in one-sidedness in the evaluation of the diversity of symbiotic bacteria. In addition, the existing evaluation methods fail to fully consider the effects of different insect-resistant genes, crop varieties, pest species and ecological environment on the interaction between transgenic crops and symbiotic bacteria of pests, thus affecting the accuracy and reliability of the evaluation results.

Therefore, it is particularly important to establish a scientific, comprehensive and accurate evaluation method to evaluate the effects of insect-resistant transgenic crops on the diversity of symbiotic bacteria in target and non-target pests. This method not only helps to deeply understand the interaction between insect-resistant transgenic crops and symbiotic bacteria of pests, but also provides scientific basis for the safety evaluation of insect-resistant transgenic crops, thus providing a strong guarantee for the sustainable development of agricultural ecosystem.

By using modern high-throughput sequencing technology, the structural and functional changes of symbiotic bacteria communities can be more comprehensively revealed, and the application of agricultural biotechnology and the assessment of environmental impact thereof can be promoted, providing important reference for future biosafety supervision and sustainable agricultural development.

### SUMMARY

An objective of the present disclosure is to provide an evaluation method for the effects of insect-resistant transgenic crops on the diversity of symbiotic bacteria in target and non-target pests to solve the limitations of existing evaluation methods, including inaccurate and incomplete evaluation results, construct a set of simple, scientific and comprehensive evaluation system, and provide a solid and reliable scientific basis for the safety evaluation of the insect-resistant transgenic crops.

The evaluation method provided by the present disclosure has wide applicability and covers various types of insect-resistant transgenic crops, including but not limited to insect-resistant transgenic crops constructed in the following ways: RNAi-mediated gene silencing, gene editing technology, and foreign gene introduction. With the help of advanced means including high-throughput sequencing technology, on the one hand, this method overcomes the disadvantages of limited detection range of traditional culture technology, and avoids the lack of symbiotic bacteria information caused by insufficient detection range; and on the other hand, it significantly reduces the workload, shortens the evaluation cycle, and has high operational feasibility and popularization and application value.

The present disclosure provides an evaluation method for the effects of insect-resistant transgenic crops on the diversity of symbiotic bacteria in target and non-target pests, which is specifically realized by the following technical solutions:
(1) planting of transgenic crops: planting insect-resistant transgenic crops and control materials thereof under controllable conditions in greenhouse, preferably under greenhouse conditions at a temperature of 25±2° C. and a relative humidity of 60±5%, and performing experiments when the crops grow to 5-8 fully unfolded leaves;
(2) selection of experimental pests: selecting target pests that are sensitive to target traits of the insect-resistant transgenic crops and non-target pests that are insensitive, preferably pests having different mouthparts types and feeding habits, and an insect age of pests in a critical harm period;
(3) treatment of pest samples: for target chewing pests, preferably feeding 2nd instar larvae (nymphs) with transgenic crops and control materials; for non-target chewing pests, preferably feeding 2nd instar larvae (nymphs) and penultimate instar larvae (nymphs) with transgenic crops and control materials; for both target and non-target sucking pests, preferably feeding adults with transgenic crops and control materials; and inoculating the pests on the insect-resistant transgenic crops and the control materials within 24 hours after molting (or molting into adults) and 12 hours after starvation treatment;
(4) collection of pest samples: collecting pest samples of the same age 48 hours after pests feed on crops; and
inoculating pests on transgenic crops and control materials within 24 hours after the pests finished molting, and collecting samples after 48 hours of feeding;
(5) extraction of sample genomic deoxyribonucleic acid (DNA): using a cetyltrimethylammonium bromide (CTAB) method to extract the genomic DNA of symbiotic bacteria in the pest samples, preferably by a soil genomic DNA extraction kit;
(6) sequencing of sample genomic DNA: sequencing the sample genomic DNA by a high-throughput sequencing method, preferably 16S ribosomal ribonucleic acid (rRNA) or metagenomic sequencing technology, and further, obtaining composition data of symbiotic bacteria community using 16S rRNA gene variable region (V3-V4 region);
(7) diversity analysis: determining the species composition, relative abundance and a/B diversity indexes of symbiotic bacteria by bioinformatics analysis, and for dominant fungi groups with relative abundance ≥3%, using analysis of variance to compare the significance of differences in symbiotic microbial communities between target and non-target pests feeding on insect-resistant transgenic crops and control materials at different taxonomic levels; and
(8) quantitative verification of key bacterial groups: for dominant bacterial groups with relative abundance ≥5% in the sequencing data, designing specific primers to determine the absolute abundance of dominant bacteria or dominant strains by absolute quantitative polymerase chain reaction (PCR), and verifying the effects of feeding the insect-resistant transgenic crops and control materials on the absolute abundance of dominant symbiotic bacteria of pests by statistical analysis.

In the present disclosure, by adopting high-throughput sequencing technology, the limitation of traditional culture technology is broken through, and the diversity of symbiotic bacteria in pests can be comprehensively and accurately detected, which provides more reliable data support for evaluating the effects of insect-resistant transgenic crops on the diversity of symbiotic bacteria in pests. The established evaluation technique method provide a scientific basis for the safety evaluation of insect-resistant transgenic crops, and can better predict the potential risks of insect-resistant transgenic crops to agricultural ecosystem, thereby taking corresponding risk prevention and control measures. In addition, this method also provides a reference for the evaluation of the effects of other transgenic crops on ecological environment, and is helpful to promote the healthy development of agricultural biotechnology and ecological environment protection.

### DETAILED DESCRIPTION

An evaluation method for the effects of insect-resistant transgenic crops on the diversity of symbiotic bacteria in target and non-target pests is described in detail below with reference to Examples.

### Example 1

This example provides an evaluation method for the effects of insect-resistant cotton on the diversity of symbiotic bacteria in a target pest Helicoverpa armigera.

### 1. Operation Steps:

(1) Planting of transgenic crops: insect-resistant transgenic cotton (transgenic Cry2Aa) and control materials were planted under greenhouse conditions at a temperature of 25±2° C. and a relative humidity of 60±5%, and the crops grew to 5-8 fully spread leaves.
(2) Selection of experimental pests: a target chewing mouthpart pest H. armigera was selected.
(3) Treatment of pest samples: 2nd instar larvae of H. armigera were inoculated on insect-resistant transgenic cotton and control materials within 24 hours after molting and 12 hours after starvation treatment.
(4) Collection of pest samples: 2nd instar samples of H. armigera were collected after 48 hours of feeding insect-resistant transgenic cotton and control materials.
(5) Extraction of sample genomic DNA: a soil genomic DNA extraction kit was used to extract genomic DNA of H. armigera.
(6) Sequencing of sample genomic DNA: the composition data of symbiotic bacteria community was obtained by sequencing a V3-V4 region of 16S rRNA.
(7) Diversity analysis: the species composition, relative abundance and diversity indexes of symbiotic bacteria were determined by bioinformatics analysis, for dominant bacterial groups with relative abundance ≥3%, the significance of differences in symbiotic bacteria communities between H. armigera feeding on insect-resistant transgenic cotton and control materials at different levels were compared by using independent sample T test.
(8) Quantitative verification of key bacterial groups: for dominant bacterial groups with relative abundance ≥5% in sequencing data, specific primers were designed to determine the absolute abundance of dominant bacteria by absolute quantitative PCR, and the effects of feeding insect-resistant transgenic cotton and control materials on the absolute abundance of dominant symbiotic bacteria of H. armigera were verified by independent sample T test analysis.

### 2. Results Analysis:

It can be seen from Table 1 that a diversity indexes (Chao1, Observedspecies, Shannon, and Simpson) of the target pest H. armigera after feeding insect-resistant transgenic cotton were significantly different from those of the target pest H. armigera after feeding control materials. This result showed that compared with the control material, the species richness and uniformity of microorganisms in H. armigera were significantly affected after feeding insect-resistant transgenic cotton.

TABLE 1
Diversity index of symbiotic bacteria after target pest
H. armigera feeding insect-resistant transgenic cotton

| | Insect-resistant | Control |
|---|---|---|
| Diversity indexes | transgenic cotton | materials |
| Chao1 | 1064.38 ± 129.8a | 509.8 ± 320.1b |
| Observedspecies | 913.9 ± 108.2a | 359.8 ± 246.1b |
| Shannon | 6.8 ± 0.2a | 1.1 ± 0.7b |
| Simpson | 1.0 ± 0.0a | 0.2 ± 0.1b |

Note:
the data in the table are mean ± standard deviation; and different lowercase letters after the same index and the same row of data indicate significant differences among different varieties (p < 0.05).

The significance of differences in the relative abundance of microorganisms in the target pest H. armigera that feeds on insect-resistant transgenic cotton at different taxonomic levels was analyzed (Table 3). The results showed that after H. armigera fed on insect-resistant transgenic cotton, the abundance of microorganisms with relative abundance ≥3% at the level of phylum, class, order, family, genus, and species showed significant differences compared with the control materials. The absolute abundance of dominant genera Enterococcus, Terrisporobacter, and Clostridium with relative abundance ≥5% was determined by absolute quantitative PCR. The results showed that the absolute abundance of these three dominant genera in H. armigera fed on insect-resistant transgenic cotton were significantly different from those fed on control materials.

TABLE 2
Relative abundance (%) of symbiotic bacteria after target pest
H. armigera feeding insect-resistant transgenic cotton

| | Insect-resistant | Control |
|---|---|---|
| Name | transgenic cotton | materials |
| Phylum level | | |
| Bacillota | 75.4 ± 2.9b | 90.7 ± 8.8a |
| Bacteroidota | 18.7 ± 2.6a | 0.9 ± 0.8b |
| Class level | | |
| Bacilli | 16.5 ± 4.8b | 89.3 ± 10.1a |
| Clostridia | 55.6 ± 11.2a | 1.0 ± 0.4b |
| Bacteroidia | 17.1 ± 3.6a | 0.8 ± 0.3b |
| Order level | | |
| Lactobacilales | 14.7 ± 2.8b | 88.9 ± 10.3a |
| Bacteroidales | 17.1 ± 3.6a | 0.8 ± 0.2b |
| Eubacteriales | 20.1 ± 5.6a | 0.3 ± 0.1b |
| Peptostreptococcales | 15.6 ± 2.5a | 0.2 ± 0.1b |
| Lachnospirales | 10.8 ± 2.6a | 0.2 ± 0.1b |
| Family level | | |
| Enterococcaceae | 6.6 ± 2.6b | 87.9 ± 10.5a |
| Peptostreptococcaceae | 15.4 ± 4.1a | 0.2 ± 0.1b |
| Lachnospiraceae | 10.7 ± 3.6a | 0.2 ± 0.1b |
| Prevotellaceae | 8.7 ± 1.1a | 0.1 ± 0.0b |
| Clostridiaceae | 7.5 ± 0.8a | 0.1 ± 0.1b |
| Genus level | | |
| Enterococcus | 6.6 ± 3.5b | 87.9 ± 10.5a |
| Terrisporobacter | 11.4 ± 3.8a | 0.1 ± 0.0b |
| Clostridium | 6.8 ± 0.9a | 0.1 ± 0.1b |
| Romboutsia | 3.9 ± 0.8a | 0.2 ± 0.1b |
| Prevotella | 3.8 ± 1.1a | 0.0 ± 0.0b |
| Species level | | |
| Enterococcus_{—}mundtii | 5.8 ± 3.8b | 87.1 ± 9.5a |
| Terrisporobacter_{—}mayombei | 11.4 ± 3.8a | 0.0 ± 0.0b |
| Clostridium_sp | 5.7 ± 0.7a | 0.0 ± 0.0b |
| Romboutsia_{—}timonensis | 3.8 ± 0.7a | 0.2 ± 0.1b |

Note:
the data in the table are mean ± standard deviation; and different lowercase letters after the same name and the same line of data indicate significant differences among different varieties (p < 0.05).

### Example 2

This example provides an evaluation method for the effects of insect-resistant transgenic cotton on the diversity of symbiotic bacteria of Aphis gossypii in non-target pests.

### 1. Operation Steps:

(1) Planting of transgenic crops: insect-resistant transgenic cotton (transgenic Cry2Aa) and control materials were planted under greenhouse conditions at a temperature of 25±2° C. and a relative humidity of 60±5%, and the crops grew to 5-8 fully spread leaves.
(2) Selection of experimental pests: a non-target prick-sucking mouthpart pest Aphis gossypii.
(3) Treatment of pest samples: the A. gossypii was inoculated into insect-resistant transgenic cotton and control materials for feeding within 24 hours after molting and 12 hours after starvation.
(4) Pest sample collection: A. gossypii samples were collected after 48 hours of feeding insect-resistant transgenic cotton and control materials.
(5) Extraction of sample genomic DNA: a soil genomic DNA extraction kit is used to extract the genomic DNA of A. gossypii.
(6) Sequencing of sample genomic DNA: the composition data of symbiotic bacteria community was obtained by sequencing a V3-V4 region of 16S rRNA.
(7) Diversity analysis: the species composition, relative abundance and diversity indexes of symbiotic bacteria were determined by bioinformatics analysis, for dominant bacterial groups with relative abundance ≥3%, the significance of differences in symbiotic bacteria communities between A. gossypii feeding on insect-resistant transgenic cotton and control materials at different levels were compared by using independent sample T test.
(8) Quantitative verification of key flora: for dominant bacterial groups with relative abundance ≥5% in sequencing data, specific primers were designed to determine the absolute abundance of dominant bacteria by absolute quantitative PCR, and the effects of feeding insect-resistant transgenic cotton and control materials on the absolute abundance of dominant symbiotic bacteria of A. gossypii were verified by independent sample T test analysis.

### 2. Results Analysis:

It can be seen from Table 3 that a diversity indexes (coverage, chao1, ace, shannon, sobs, and simpson) of A. gossypii after feeding insect-resistant transgenic cotton were not significantly different from those of A. gossypii after feeding control materials. This result showed that compared with the control material, the species richness and uniformity of microorganisms in A. gossypii were significantly affected after feeding insect-resistant transgenic cotton.

TABLE 3
Diversity index of symbiotic bacteria after target pest
A. gossypii feeding insect-resistant transgenic cotton

| | Insect-resistant | Control |
|---|---|---|
| Diversity indexes | transgenic cotton | materials |
| Coverage | 1.0 ± 0.0a | 1.0 ± 0.0a |
| Chao1 | 89.3 ± 7.8a | 84.0 ± 5.7a |
| Ace | 89.2 ± 7.8a | 84.0 ± 5.7a |
| Shannon | 3.4 ± 0.2a | 3.3 ± 0.2a |
| Sobs | 89.2 ± 7.8a | 84.0 ± 5.7a |
| Simpson | 0.1 ± 0.0a | 0.1 ± 0.0a |

Note:
the data in the table are mean ± standard deviation; and same lowercase letters after the same index and the same row of data indicate significant differences among different varieties (p < 0.05).

The significance of differences in the relative abundance of microorganisms in the non-target pest A. gossypii that feeds on insect-resistant transgenic cotton at different taxonomic levels was analyzed (Table 4). The results showed that after A. gossypii fed on insect-resistant transgenic cotton, the abundance of microorganisms with relative abundance ≥3% at the level of phylum, class, order, family, genus, and species showed no significant difference compared with the control materials. The absolute abundance of dominant genera Acinetobacter, Pelomonas, Delftia, and Paracidovorax with relative abundance ≥5% was determined by absolute quantitative PCR. The results showed that the absolute abundance of these four dominant genera in A. gossypii fed on insect-resistant transgenic cotton were not significantly different from those fed on control materials.

TABLE 4
Relative abundance (%) of symbiotic bacteria after target pest
A. gossypii feeding insect-resistant transgenic cotton

| | Insect-resistant | Control |
|---|---|---|
| Name | transgenic cotton | materials |
| Phylum level | | |
| Pseudomonadota | 92.8 ± 3.8a | 89.6 ± 3.8a |
| Bacillota | 3.9 ± 2.6a | 6.8 ± 3.9a |
| Class level | | |
| Gammaproteobacteria | 60.6 ± 6.4a | 50.1 ± 13.0a |
| Betaproteobacteria | 23.3 ± 5.4a | 34.0 ± 13.2a |
| Alphaproteobacteria | 8.8 ± 2.8a | 5.5 ± 1.7a |
| Bacilli | 3.8 ± 2.6a | 6.9 ± 3.8a |
| Order level | | |
| Moraxellales | 53.3 ± 5.6a | 40.9 ± 11.6a |
| Burkholderiales | 22.7 ± 5.2a | 33.6 ± 13.1a |
| Bacillales | 3.5 ± 2.8a | 6.3 ± 3.5a |
| Pseudomonadales | 5.1 ± 0.7a | 4.3 ± 1.8a |
| Hyphomicrobiales | 3.3 ± 0.6a | 2.5 ± 0.6a |
| Sphingomonadales | 3.0 ± 1.8a | 1.0 ± 0.9a |
| Family level | | |
| Moraxellaceae | 53.3 ± 5.6a | 40.9 ± 11.6a |
| Comamonadaceae | 14.3 ± 3.3a | 17.3 ± 4.9a |
| Burkholderiaceae | 5.9 ± 2.3a | 4.8 ± 1.6a |
| Pseudomonadaceae | 5.1 ± 0.7a | 4.3 ± 1.8a |
| Bacillaceae | 2.8 ± 2.3a | 3.6 ± 3.5a |
| Genus level | | |
| Acinetobacter | 53.3 ± 5.6a | 40.9 ± 11.6a |
| Pelomonas | 8.9 ± 2.8a | 8.0 ± 3.6a |
| Delftia | 9.8 ± 0.6a | 9.8 ± 1.8a |
| Paracidovorax | 9.2 ± 0.1a | 9.2 ± 3.3a |
| Pseudomonas | 4.7 ± 0.7a | 4.3 ± 1.9a |
| Burkholderia | 3.2 ± 1.7a | 3.1 ± 1.1a |
| Buchnera | 4.3 ± 1.3a | 4.0 ± 1.1a |
| Species level | | |
| Acinetobacter_{—}guillouiae | 24.2 ± 2.3a | 18.9 ± 6.0a |
| Acinetobacter_{—}bereziniae | 22.1 ± 2.7a | 17.5 ± 4.9a |
| Pelomonas_{—}saccharophila | 8.8 ± 2.8a | 8.0 ± 3.6a |
| Delftia_sp._BR1 | 4.7 ± 0.3a | 5.8 ± 2.8a |
| Burkholderia_sp._Bp8984 | 3.9 ± 1.3a | 3.0 ± 1.0a |
| Acinetobacter_{—}johnsonii | 3.4 ± 1.2a | 2.0 ± 0.8a |

Note:
the data in the table are mean ± standard deviation; and same lowercase letters after the same index and the same row of data indicate significant differences among different varieties (p ≥ 0.05).

### Example 3

This example provides an evaluation method for effects of insect-resistant transgenic soybeans on the diversity of symbiotic bacteria in vivo of non-target pest H. armigera.

### 1. Operation Steps:

(1) Planting of transgenic crops: insect-resistant transgenic soybeans and control materials were planted under greenhouse conditions at a temperature of 25±2° C. and a relative humidity of 60±5%, and the crops grew to 5-8 fully spread leaves.
(2) Selection of experimental pests: a non-target pest chewing mouthpart pest H. armigera.
(3) Treatment of pest samples: 2nd and 5th instar larvae of H. armigera were inoculated on insect-resistant transgenic soybeans and control materials within 24 hours after molting and 12 hours after starvation treatment.
(4) Pest sample collection: H. armigera samples were collected after 48 hours of feeding insect-resistant transgenic soybeans and control materials.
(5) Extraction of sample genomic DNA: a soil genomic DNA extraction kit is used to extract the genomic DNA of H. armigera.
(6) Sequencing of sample genomic DNA: the composition data of symbiotic bacteria community was obtained by sequencing a V3-V4 region of 16S rRNA.
(7) Diversity analysis: the species composition, relative abundance and diversity indexes of symbiotic bacteria were determined by bioinformatics analysis, for dominant bacterial groups with relative abundance ≥3%, the significance of differences in symbiotic bacteria communities between H. armigera feeding on insect-resistant transgenic soybeans and control materials at different levels were compared by using independent sample T test.
(8) Quantitative verification of key flora: for dominant bacterial groups with relative abundance ≥5% in sequencing data, specific primers were designed to determine the absolute abundance of dominant bacteria by absolute quantitative PCR, and the effects of feeding insect-resistant transgenic soybeans and control materials on the absolute abundance of dominant symbiotic bacteria of H. armigera were verified by independent sample T test analysis.

### 2. Results Analysis:

(1) Effects of Insect-Resistant Transgenic Soybeans on the Diversity of Symbiotic Bacteria in 2nd Instar Larvae of H. armigera

It can be seen from Table 5 that a diversity indexes (coverage, chao1, ace, shannon, sobs, and simpson) of the 2nd instar larvae of H. armigera after feeding insect-resistant transgenic soybean were not significantly different from those of the 2nd instar larvae of H. armigera after feeding control materials. This result showed that compared with the control material, the species richness and uniformity of microorganisms in the 2nd instar larvae of H. armigera were not significantly affected after feeding on insect-resistant transgenic soybeans.

TABLE 5
Diversity index of symbiotic bacteria after 2nd instar larvae
of H. armigera feeding insect-resistant transgenic soybeans

| | Insect-resistant | Control |
|---|---|---|
| Diversity index | transgenic soybeans | Material |
| Coverage | 1.0 ± 0.0a | 1.0 ± 0.0a |
| Chao1 | 64.2 ± 7.3a | 60.5 ± 10.0a |
| Ace | 64.8 ± 7.0a | 60.7 ± 10.0a |
| Shannon | 2.5 ± 0.1a | 2.5 ± 0.1a |
| Sobs | 64.0 ± 7.5a | 60.5 ± 9.9a |
| Simpson | 0.2 ± 0.0a | 0.2 ± 0.0a |

Note:
the data in the table are mean ± standard deviation; and same lowercase letters after the same index and the same row of data indicate significant differences among different varieties (p ≥ 0.05).

The significance of differences in the relative abundance of microorganisms in the non-target pest 2nd instar larvae of H. armigera that feeds on insect-resistant transgenic soybeans at different taxonomic levels was analyzed (Table 6). The results showed that after the 2nd instar larvae of H. armigera fed on insect-resistant transgenic soybeans, the abundance of microorganisms with relative abundance ≥3% at the level of phylum, class, order, family, genus, and species showed no significant difference compared with the control materials. The absolute abundance of dominant genera Enterococcus, Mammaliicoccus, and Enterobacter with relative abundance ≥5% was determined by absolute quantitative PCR. The results showed that the absolute abundance of these three dominant genera in the 2nd instar larvae of H. armigera fed on insect-resistant transgenic soybeans were not significantly different from those fed on control materials.

TABLE 6
Relative abundance (%) of symbiotic bacteria after 2nd instar larvae
of H. armigera feeding on insect-resistant transgenic soybeans

| | Insect-resistant | Control |
|---|---|---|
| Name | transgenic soybeans | Material |
| Phylum level | | |
| Bacillota | 92.9 ± 2.3a | 94.2 ± 0.9a |
| Pseudomonadota | 5.0 ± 1.5a | 3.7 ± 0.5a |
| Class level | | |
| Bacilli | 92.9 ± 2.3a | 94.2 ± 0.9a |
| Gammaproteobacteria | 4.5 ± 1.4a | 3.1 ± 0.6a |
| Order level | | |
| Lactobacilales | 89.2 ± 3.2a | 89.3 ± 1.1a |
| Bacillales | 3.6 ± 0.9a | 4.8 ± 0.8a |
| Enterobacterales | 4.4 ± 1.5a | 3.2 ± 0.6a |
| Family level | | |
| Enterococcaceae | 86.0 ± 4.3a | 87.5 ± 1.0a |
| Staphylococcaceae | 3.6 ± 0.9a | 4.6 ± 1.1a |
| Enterobacteriaceae | 4.3 ± 1.4a | 3.0 ± 0.6a |
| Genus level | | |
| Enterococcus | 86.0 ± 4.3a | 87.5 ± 1.0a |
| Mammaliicoccus | 3.6 ± 0.9a | 4.5 ± 1.0a |
| Enterobacter | 3.8 ± 1.2a | 2.5 ± 0.7a |
| Species level | | |
| Enterococcus_{—}casseliflavus | 48.4 ± 2.9a | 46.7 ± 2.1a |
| Enterococcus_{—}mundtii | 17.2 ± 1.1a | 19.9 ± 1.2a |
| Enterococcus_{—}innesii | 4.8 ± 0.5a | 3.3 ± 2.2a |
| Mammaliicoccus_{—}sciuri | 3.6 ± 0.9a | 4.5 ± 1.0a |
| Enterococcus_sp._DIV0242_7C1 | 3.2 ± 1.1a | 4.7 ± 0.5a |

Note:
the data in the table are mean ± standard deviation; and same lowercase letters after the same index and the same row of data indicate significant differences among different varieties (p ≥ 0.05).

(2) Effects of Insect-Resistant Transgenic Soybeans on the Diversity of Symbiotic Bacteria in 5th Instar Larvae of H. armigera

It can be seen from Table 7 that a diversity indexes (coverage, chao1, ace, shannon, sobs, and simpson) of the 5th instar larvae of H. armigera after feeding insect-resistant transgenic soybean were not significantly different from those of the 5th instar larvae of H. armigera after feeding control materials. This result showed that compared with the control material, the species richness and uniformity of microorganisms in the 5th instar larvae of H. armigera were not significantly affected after feeding on insect-resistant transgenic soybeans.

TABLE 7
Diversity index of symbiotic bacteria after 5th instar larvae
of H. armigera feeding insect-resistant transgenic soybeans

| | Insect-resistant | Control |
|---|---|---|
| Diversity index | transgenic soybeans | Material |
| Coverage | 1.0 ± 0.0a | 1.0 ± 0.0a |
| Chao1 | 64.2 ± 7.3a | 60.5 ± 10.0a |
| Ace | 64.8 ± 7.0a | 60.7 ± 10.0a |
| Shannon | 2.5 ± 0.1a | 2.5 ± 0.1a |
| Sobs | 64.0 ± 7.5a | 60.5 ± 9.9a |
| Simpson | 0.2 ± 0.0a | 0.2 ± 0.0a |

Note:
the data in the table are mean ± standard deviation; and same lowercase letters after the same index and the same row of data indicate significant differences among different varieties (p ≥ 0.05).

The significance of differences in the relative abundance of microorganisms in the non-target pest 5th instar larvae of H. armigera that feeds on insect-resistant transgenic soybeans at different taxonomic levels was analyzed (Table 8). The results showed that after the 5th instar larvae of H. armigera fed on insect-resistant transgenic soybeans, the abundance of microorganisms with relative abundance ≥3% at the level of phylum, class, order, family, genus, and species showed no significant difference compared with the control materials. The absolute abundance of dominant bacterial genus Enterococcus with relative abundance ≥5% was determined by absolute quantitative PCR. The results showed that the absolute abundance of this dominant genera in the 5th instar larvae of H. armigera fed on insect-resistant transgenic soybeans were not significantly different from those fed on control materials.

TABLE 8
Relative abundance (%) of symbiotic bacteria after 5th instar larvae
of H. armigera feeding on insect-resistant transgenic soybeans

| | Insect-resistant | Control |
|---|---|---|
| Name | transgenic soybeans | Material |
| Phylum level | | |
| Bacillota | 99.2 ± 0.5a | 99.5 ± 0.1a |
| Class level | | |
| Bacilli | 99.1 ± 0.5a | 99.4 ± 0.1a |
| Order level | | |
| Lactobacilales | 98.1 ± 0.3a | 98.0 ± 0.2a |
| Family level | | |
| Enterococcaceae | 97.6 ± 0.5a | 96.6 ± 0.9a |
| Genus level | | |
| Enterococcus | 97.6 ± 0.5a | 96.6 ± 0.9a |
| Species level | | |
| Enterococcus_{—}casseliflavus | 77.4 ± 1.3a | 79.3 ± 0.8a |
| Enterococcus_{—}mundtii | 5.1 ± 0.5a | 4.3 ± 0.3a |
| Enterococcus_{—}innesii | 6.5 ± 0.3a | 6.4 ± 0.1a |
| Enterococcus_{—}casseliflavus | 77.4 ± 1.3a | 79.3 ± 0.8a |

Note:
the data in the table are mean ± standard deviation; and same lowercase letters after the same index and the same row of data indicate significant differences among different varieties (p ≥ 0.05).

The above instances are merely illustrative of preferred embodiments of the present disclosure, and do not limit the scope of the present disclosure. On the premise of not departing from the design idea of the present disclosure, various modifications, modifications or improvements made by those skilled in the art to the technical solutions of the present disclosure shall be included within the scope of protection defined by the claims of the present disclosure.

1. An evaluation method for the effects of insect-resistant transgenic crops on the diversity of symbiotic bacteria in target and non-target pests, comprising the steps of:
(1) planting of transgenic crops: planting insect-resistant transgenic crops and control materials thereof under controllable conditions in greenhouse, and performing experiments when the crops grow to 5-8 fully unfolded leaves;
(2) selection of experimental pests: selecting target pests that are sensitive to target traits of the insect-resistant transgenic crops and non-target pests that are insensitive, preferably an insect age of pests in a critical harm period;
(3) treatment of pest samples: inoculating the pests on the insect-resistant transgenic crops and the control materials within 24 hours after molting (or molting into adults) and 12 hours after starvation treatment;
(4) collection of pest samples: collecting pest samples of the same age 48 hours after pests feed on crops;
(5) extraction of sample genomic deoxyribonucleic acid (DNA): taking the genomic DNA of symbiotic bacteria in the pest samples;
(6) sequencing of sample genomic DNA: sequencing the sample genomic DNA by a high-throughput sequencing method to obtain composition data of symbiotic bacteria community;
(7) diversity analysis: determining the species composition, relative abundance and a/B diversity indexes of symbiotic bacteria by bioinformatics analysis, and for dominant fungi groups with relative abundance ≥3%, using analysis of variance to compare the significance of differences in symbiotic microbial communities between target and non-target pests feeding on insect-resistant transgenic crops and control materials at different taxonomic levels; and
(8) quantitative verification of key bacterial groups: for dominant bacterial groups with relative abundance ≥5% in the sequencing data, designing specific primers to determine the absolute abundance of dominant bacteria or dominant strains by absolute quantitative polymerase chain reaction (PCR), and verifying the effects of feeding the insect-resistant transgenic crops and control materials on the absolute abundance of dominant symbiotic bacteria of pests by statistical analysis.

2. The evaluation method according to claim 1, wherein the insect-resistant transgenic crops are constructed by ribonucleic acid interference (RNAi)-mediated gene silencing, gene editing technology, and foreign gene introduction.

3. The evaluation method according to claim 1, wherein in step (2), pests having different mouthparts types and feeding habits are selected as the target pests and the non-target pests.

4. The evaluation method according to claim 1, wherein in step (3), for target chewing pests, 2nd instar larvae (nymphs) are preferred to feed on transgenic crops and control materials; for non-target chewing pests, 2nd instar larvae (nymphs) and the penultimate instar larvae (nymphs) are preferred to feed on transgenic crops and control materials; and for both target and non-target sucking pests, adults are preferred to feed on transgenic crops and control materials.

5. The evaluation method according to claim 1, wherein the extraction of sample genomic DNA in step (5) is performed using a cetyltrimethylammonium bromide (CTAB) method, preferably by a soil genomic DNA extraction kit.

6. The evaluation method according to claim 1, wherein in step (6), 16S ribosomal ribonucleic acid (rRNA) or metagenomic sequencing technology is preferably used, and further, 16S rRNA gene variable region sequencing is preferably used to obtain the composition data of the commensal bacteria community.
